(19) United States
(12) Patent Application Publication
(10) Pub. No.: US 2023/0230676 A1
(43) Pub. Date: Jul. 20, 2023

(54) Contingency Management Systems and Methods with Gamification

(71) Applicant: Steven Jenkins, Rindge, NH (US)

(72) Inventor: Steven Jenkins, Rindge, NH (US)

(21) Appl. No.: 17/578,193

(22) Filed: Jan. 18, 2022

(51) Int. Cl. G16H 20/70; G06Q 30/02

(52) U.S. Cl. CPC G16H 20/70; G06Q 30/0209

(57) ABSTRACT

Methods and systems of contingency management that utilize games of chance to reinforce positive behaviors in participants are disclosed herein. The utilization of games to reinforce positive behaviors may also be referred to as gamification. The contingency management systems and methods with gamification disclosed herein may reduce costs and increase engagement for participants in a contingency management program that utilizes such systems or methods.

### BACKGROUND

### Technical Field

The present disclosure relates generally to contingency management systems and methods with gamification. More particularly, the present disclosure relates to opioid addiction recovery support contingency management systems and methods that utilize gamification to improve outcomes for patients.

### Description of Related Art

Contingency management refers to a behavioral therapy in which individuals are reinforced or rewarded for evidence of positive behavioral change. The general idea of using contingency management principles to create a treatment plan for substance abuse and other related disorders is known. Current known methods of contingency management, particularly those in the medical field, suffer from numerous problems, including, but not limited to, high rates of program dropouts, patient relapses, admission, readmission, and overdose incidents. There are a plethora of potential solutions to these problems; however, the outcomes of the application of such solutions are unpredictable due, at least in part, to the difficulty of predicting human behavior.

Therefore, what is needed is a system or method of contingency management with gamification having the following characteristics and benefits over the prior art.

### SUMMARY

The subject matter of this application may involve, in some cases, interrelated products, alternative solutions to a particular problem, and/or a plurality of different uses of a single system or article.

In one aspect, a contingency management system is disclosed. In this aspect the contingency management system comprises a first server; a contingency management engine; and a computer memory, wherein the computer memory is configured to store program instructions executable by at least one processor.

In another aspect, the contingency management engine is operable to at least conduct the steps of sending event information to a first user device; and sending a chance for rewards to a second user device.

It should be expressly understood that the various physical elements of the present disclosure summarized and further disclosed herein may be of varying sizes, shapes, or otherwise dimensions and made from a variety of different materials or methods of manufacture without straying from the scope of the present disclosure.

### BRIEF DESCRIPTION OF THE DRAWINGS

FIG. 1 provides a perspective view of an embodiment of the present disclosure.

FIG. 2 provides a perspective view of an embodiment of a user interface for a mobile device according to the present disclosure.

FIG. 3 provides a perspective view of another embodiment of a user interface for cashing in rewards for electronic gift cards according to the present disclosure.

FIG. 4 provides a perspective view of an embodiment of a user interface for tracking rewards according to the present disclosure.

FIG. 5 provides a perspective view of another embodiment of the present disclosure.

FIG. 6 provides a perspective view of yet another embodiment of the present disclosure.

FIG. 7 provides a perspective view of an embodiment of a user interface of an administrative user device according to the present disclosure.

FIG. 8 provides a perspective view of an embodiment of an administrative user interface for configuring a contingency management rewards engine according to the present disclosure.

FIG. 9 provides a perspective view of an embodiment of inputs for an algorithm that determines the effectiveness of a configured contingency management program of the present disclosure.

FIG. 10 provides a perspective view of embodiments of user interfaces for games according to the present disclosure.

FIG. 11 provides a perspective view of embodiments of user interfaces for tiered gaming according to the present disclosure.

FIG. 12 provides a perspective view of an embodiment of a user interface displaying badge rewards according to the present disclosure.

### DETAILED DESCRIPTION

The detailed description set forth below in connection with the appended drawings is intended as a description of presently preferred embodiments of the invention and does not represent the only forms in which the present disclosure may be constructed and/or utilized. The description sets forth the functions and the sequence of steps for constructing and operating the invention in connection with the illustrated embodiments.

Generally, the present disclosure concerns methods and systems of contingency management that utilize games of chance to reinforce positive behaviors in participants in a contingency management program. The utilization of games to reinforce positive behaviors may also be referred to as gamification. The contingency management systems and methods with gamification disclosed herein may reduce costs and increase engagement for participants in a contingency management program that utilizes such systems or methods. While the unique methods and systems disclosed herein may be generally applied to any contingency management program, the present disclosure may more preferably be used to target negative behaviors for change in patients addicted to opioids or other stimulants.

Some embodiments of the present disclosure may include a non-transitory computer readable medium or memory, wherein the medium or memory contains program instructions allowing and instructing a processor to execute the steps required during the operation of the contingency management system.

This non-transitory computer readable memory may be stored within a computer or other similar computerized device utilizing a processor, such as a mobile device. Furthermore, in some embodiments, the contingency management system may be accessible through an electronic communication system, such as a network connection, for example, the Internet.

Accordingly, many embodiments of the contingency management system contemplated herein may be carried out on a computerized and networked platform, which may allow both administrators and participants to log in via their own computers or computerized devices to a system with memory storing the data relating to the system, user interface information, and having local or networked access to a server or database containing information regarding the variables of a contingency management engine, an administrative dashboard, or a contingency management application accessible by patients.

In many embodiments, the contingency management system may comprise three primary elements: (1) a contingency management engine that controls the variables for a contingency management program; (2) an administrative dashboard; and (3) a contingency management application. The contingency management engine enables the design and deployment of contingency management programs around certain variables that will be discussed further herein. The contingency management engine may be stored on either an administrative user device's system memory, a server and/or a database within the server. In some embodiments, the contingency management engine may be run on a processor of an administrative user device. Depending on the particular embodiment, the contingency management engine may provide the ability to set a number of different targeted behavioral events for change as well as the length of the program, overall budget, number of events, type of events, and budget by event type.

In some embodiments, the contingency management system may be operable to present an administrative dashboard view of individual patient's contingency management program with milestones, chances, games, or rewards used or won. The administrative dashboard may allow user to view actionable data such as analytics and risk scores with decision support.

The dashboard may also allow administrative users to schedule and run individual or group telehealth appointments for patients to attend through their contingency management application.

In some embodiments, the contingency management application may be an application for a mobile user device, wherein patients are provided with a user interface displaying their individual contingency management program with milestones, chances, games, or rewards used or won. Furthermore, in some embodiments, the application may comprise a digital wallet to hold patients' rewards or prizes. Either the administrative dashboard or the application may be operable to present surveys or a comments section to users in order to allow the contingency management system to be constantly improved with user feedback.

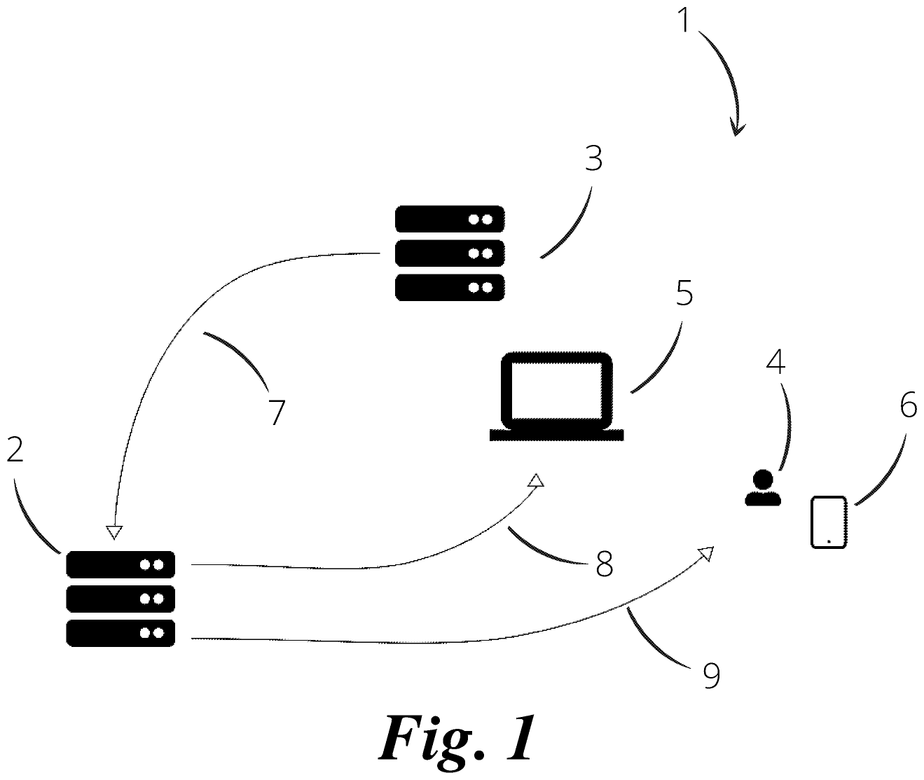

Turning now to FIG. 1, which shows a perspective view of one embodiment of a contingency management system 1. In this embodiment, the system 1 utilizes a first server 2 and a second server 3. In this embodiment, the first server 2 stores data or information regarding the variables of a contingency management program. In other words, the first server 2 runs the contingency management engine for the system 1. The second server 3 stores data or information regarding specific behavioral events for participants in the contingency management program. For example, in this embodiment, a patient 4 is one of the participants in the contingency management program, and the specific behavioral event data stored in the second server 3 may be data regarding, for example, whether the patient 4 filled a prescription, or whether the patient's 4 urine toxicology (“UTOX”) results were analyte positive or negative. In other words, in this embodiment, the second server 3 is the server for an electronic health record (“EHR”) database.

In the embodiment shown in FIG. 1, the contingency management system 1 is operable to validate events and generate reward chances based on the variables of the contingency management engine. The contingency management system 1 accomplishes this generally through communication of information between the first server 2, the second server 3, and a plurality of user devices. In this embodiment, the plurality of user devices comprises an administrative user device 5 and a mobile user device 6. The validation of events is accomplished by the second server 3 being operable to send 7 information regarding specific behavioral events to the first server 2 upon request. For example, the second server 3 may send 7 information confirming that the patient 4 attended their appointment or that the UTOX results for the patient 4 were analyte negative. The first server 2 is then operable to send 8 the event information to the administrative user device 5. In other words, the event information displayed on the administrative user device 5 is validated by the EHR database of the second server 3.

The electronic validation of events through a second server 3 solves a general problem with contingency management programs. In some contingency management programs, behavioral events are participant reported, and thus, the occurrence of events is open to manipulation. Utilizing a server containing an EHR database or other similar stored information from third party systems or clinical teams to validate events removes or, at the very least, significantly reduces chances for manipulation and ensures the overall effectiveness of the contingency management program.

As further shown in FIG. 1 and similar to the electronic validation of events, the first server 2 is also operable to generate and send 9 rewards chances to the mobile user device 6 based on whether certain targeted behaviors, pre-defined by the contingency management engine, occurred in the event. For example, in this embodiment, the patient 4 owns the mobile user device 6, and the rewards chances are sent to the contingency management application displayed on the user interface of the mobile user device 6. In this embodiment, a targeted behavior could be, for example, analyte negative UTOX results, and the contingency management engine could be pre-set to, for example, generate a ninety percent (90%) chance for a reward based on negative UTOX results. In other words, in this embodiment, if the UTOX results for a patient 4 are electronically validated to be analyte negative, the first server 2 is operable to send a ninety percent (90%) chance for a reward to the contingency management application on the mobile user device 6.

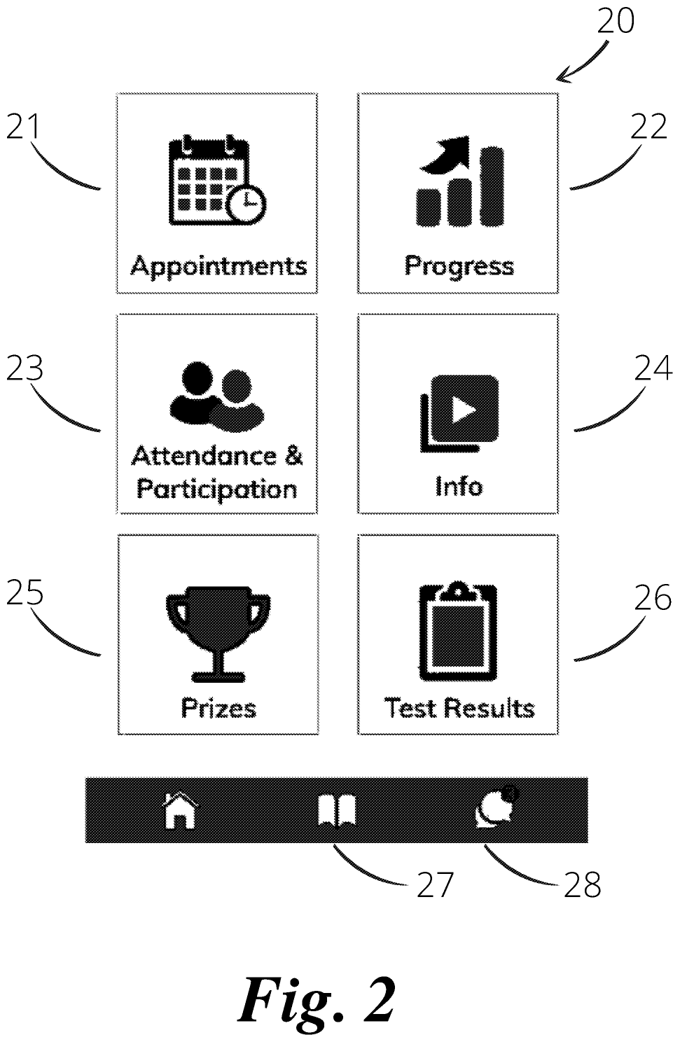

FIG. 2 shows a perspective view of an embodiment of a user interface for the contingency management application 20 displayed on a mobile user device 6. In this embodiment, the application 20 is operable to display various tabs for patient interaction and navigation, including an appointment tab 21, a progress tab 22, an attendance and participation tab 23, an information tab 24, a prizes tab 25, and a test results tab 26, a journal tab 27, and a messaging tab 28. Each of the various tabs corresponds to the functions of the application 20.

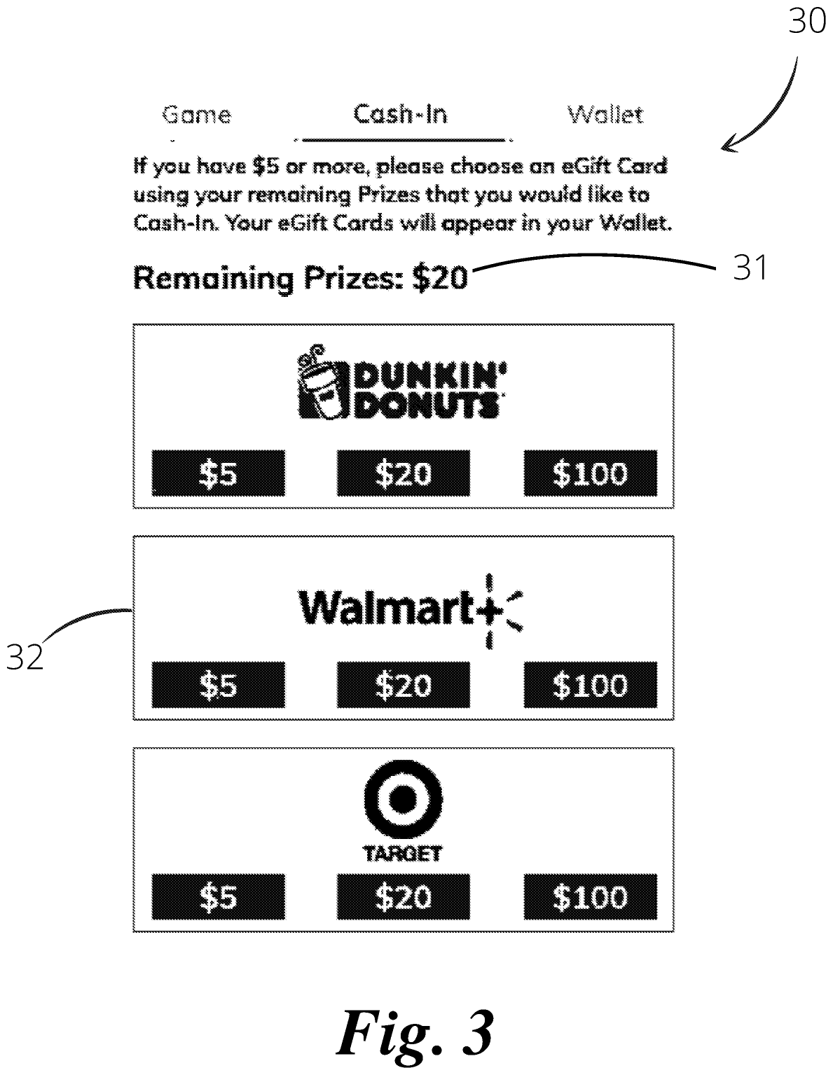

For example, in the embodiment shown in FIG. 2, the application 20 is operable to keep a calendar of appointments with reminders for the patient, and the application 20 is further operable to display said calendar with appointments and reminders, which the patient may access through the appointment tab 21. The progress tab 22 allows patients to plot and track their progress and successes. The attendance and participation tab 23 allows patients to track their attendance of appointments or meetings. The information tab 24 provides patients with general information about the application or the contingency management program. The prizes tab 25 allows patients to cash in earned rewards for prizes, including, but not limited to, electronic gift cards, as shown in FIG. 3, debit cards with real time auto top ups, redeemable vouchers, or curated items and/or progress towards same in a digital shop.

FIG. 3 provides a perspective view of an embodiment of a user interface 30 for cashing in rewards 31 for electronic gift cards 32. In this embodiment of a user interface 30, patients can view their rewards 31 and then redeem said rewards for prizes in the form of electronic gift cards 32 from approved vendors, including, but not limited to, Dunkin Donuts®, Walmart®, or Target®, as shown in FIG. 3. This embodiment solves a problem with contingency management systems, particularly systems that reward participants with cash prizes. In the context of opioid or other stimulant addiction recovery contingency management systems, cash prizes generate the risk that participants will use the cash prizes to purchase opioids or other stimulants, which may lead to relapses, admission, readmission, overdose incidents, or other negative outcomes. By providing participants electronic delivery of gift cards on their mobile user device 6, the risk that participants will use their prizes to resort to prior negative behaviors is eliminated.

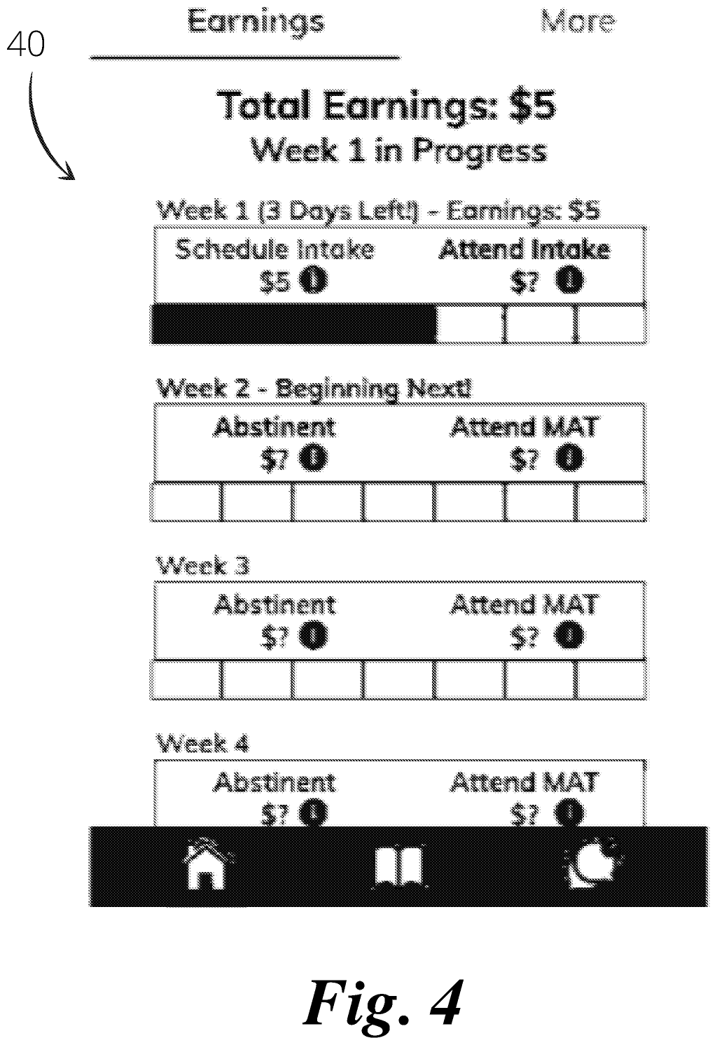

FIG. 4 provides a perspective view of an embodiment of a user interface 40 for tracking rewards and progress. This embodiment also provides patients with a way to see their opportunities for future rewards. In many contingency management systems, participants are not able to track their progress as they receive rewards for positive behavioral change throughout the program. This embodiment of a user interface 40 solves this problem by allowing participants to track their progress to see their rewards for positive behavioral events, which provides participants with immediate positive reinforcement and gives them a sense of achieving both short-term and long-term goals. Showing patients opportunities for future rewards may also increase patient engagement with the contingency management program.

Referring back to FIG. 2, the test results tab 26 allows patients to view any test results, such as UTOX results for opioids or other stimulants. The viewing of analyte negative UTOX results may also provide patients with a sense of achieving both short-term and long-term goals, which encourages patients to further adhere to the contingency management program or treatment. The journal tab 27 allows patients to both keep and share a daily journal. The daily journal allows patients to securely log and share thoughts and stay connected and supported between visits. In this embodiment, the messaging tab 28 allows for patients to participate in secure messaging with clinicians in addition to anonymous messaging with peers. This allows clinicians to directly participate in and aid patients in recovery throughout the program. It also allows patients to receive recovery support in an anonymous, secure, and stigma-free environment to eventually build a network of sober friends.

All of the aforementioned aspects of the present disclosure solve the general problem of low participant adherence to a contingency management program and significantly reduce rates of participants dropping out of the program. However, the messaging tab 28 in particular also solves a problem specific to the field of computers and other electronic devices. In many contingency management systems a messaging function is either not provided or, if provided, is available as a separate application. By providing the messaging function in a messaging tab 28 on the contingency management application 20, patients do not need to navigate away from the application 20 to receive immediate support from clinicians or peers through a messaging function on a separate application. This solves the problem of limited application functionality in the field of computers and other electronic devices.

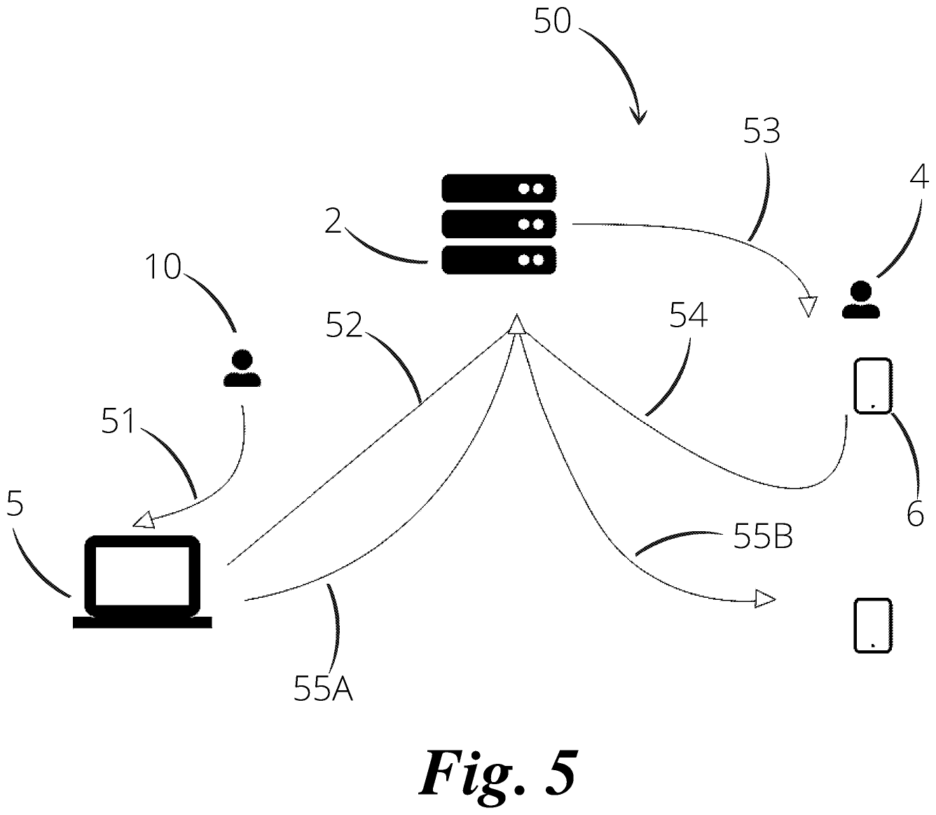

Turning now to FIG. 5, which provides a perspective view of an embodiment of a messaging process 50 within the contingency management system 1. In this embodiment, an administrative user 10 initiates 51 the sending of a message to a patient 4 via an administrative user device 5. The administrative user device is operable to send 52 the message from the administrative user device 5 to a first server 2. The first server 2 then sends 53 the message to the patient 4, which the patient 4 receives on their mobile user device 6. The patient 4 may then respond to the message through their mobile user device 6, which sends 54 the response to the first server 2. In this embodiment and upon receipt of the patient's response, the first server 2 is then operable to both send 55A the patient's 4 response to the administrative user device 5 and also generate rewards chances for prizes, which the first server 2 is then operable to send 55B to the mobile user device 6. In this way, the first server 2 is able to validate the positive behavioral event of the patient 4 responding to a message from an administrative user 10 or other users, such as clinicians or other health care support staff, and the first server 2 is also able to generate rewards chances to reinforce the positive behavior. In this embodiment, the first server 2 is also the server that runs the contingency management engine for the contingency management program.

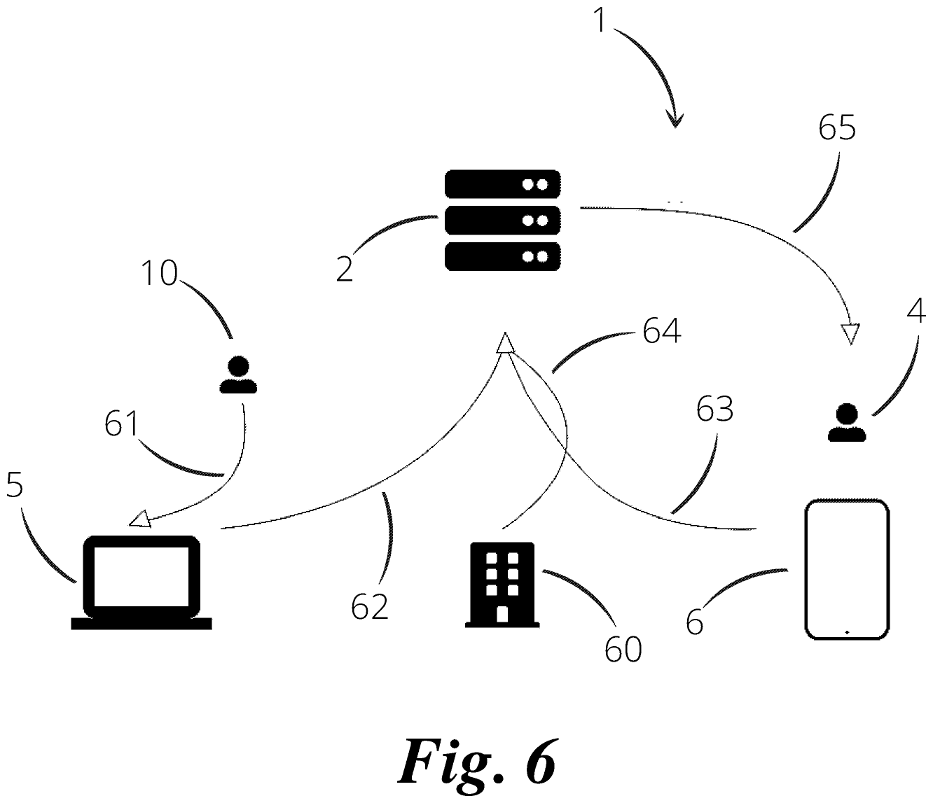

As shown in the embodiment of the system 1 depicted in FIG. 6, the first server 2 is also operable to validate events and generate rewards chances in response to events that are not a patient 4 responding to a message. For example, in this embodiment, the system 1 is operable to validate a patient's 4 attendance of an appointment and generate rewards chances based on this positive behavior. The electronic validation of appointment attendance is accomplished generally by communication between an administrative user device 5, a first server 2, which in this embodiment runs the contingency management engine for the system 1, and a mobile user device 6. An administrative user 10 initiates the validation process by entering 61 appointment information on the administrative user device 5, which in turn sends 62 the appointment information to the first server 2. In this embodiment, the appointment information is the date, time, and location 60 of the appointment. The patient 4 may then attend the appointment, and the patient's 4 mobile user device 6 confirms 63 attendance of the appointment through a geographic information system (“GIS”). The first server 2 confirms the patient's 4 attendance of the appointment by cross checking 64 the GIS information for the geographic location of the mobile user device 6 and the GIS information for the geographic location of the appointment location 60 in addition to the date and time information for the appointment in the first server 2 and/or a database within the first server 2. If the GIS and other appointment information match, the first server 2 is then operable to generate and send 65 rewards chances for prizes to the mobile user device.

Using GIS to validate the patient's attendance of an appointment solves the problem of a patient self-reporting false information in order to receive prizes. In other words, the GIS validation of a patient's appointment attendance removes or, at the very least, significantly reduces chances for manipulation and ensures the overall effectiveness and fidelity of the contingency management program.

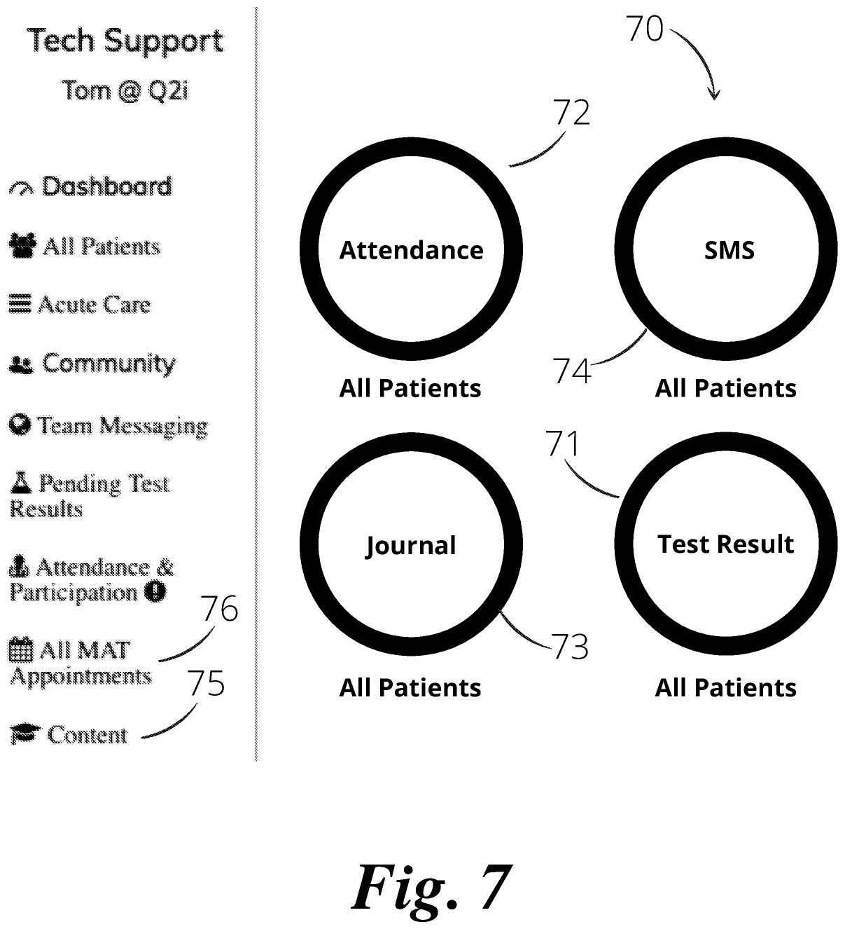

Turning now to FIG. 7, which shows a perspective view of an embodiment of a user interface for an administrative dashboard 70. In this embodiment, the administrative dashboard 70 allows administrative users to view patient test results through a test results tab 71. The administrative dashboard 70 also allows administrative users to track patient attendance of appointments through the appointments tab 72. The journal tab 73 allows management of patient feedback and emotions by viewing patients' self-reporting in their electronic journals. In this embodiment, administrative users can also send and receive secure messages from patients via the message tab 74. In this embodiment, administrators can send and receive messages to and from patients through a short message service (“SMS”), electronic mail (“e-mail”), or other forms of electronic communication. The content distribution tab 75 allows administrative users to manage and/or send educational materials to participants. In some embodiments, the educational material may be quizzes published in a contingency management application on patients' mobile devices, wherein the system may be operable to track the viewing of said material and send rewards chances or opportunities to play games to patients' devices based on patients' scores or participation with said material. The appointment tab 76 allows administrative users to manage calendars and appointments to patients' computerized devices.

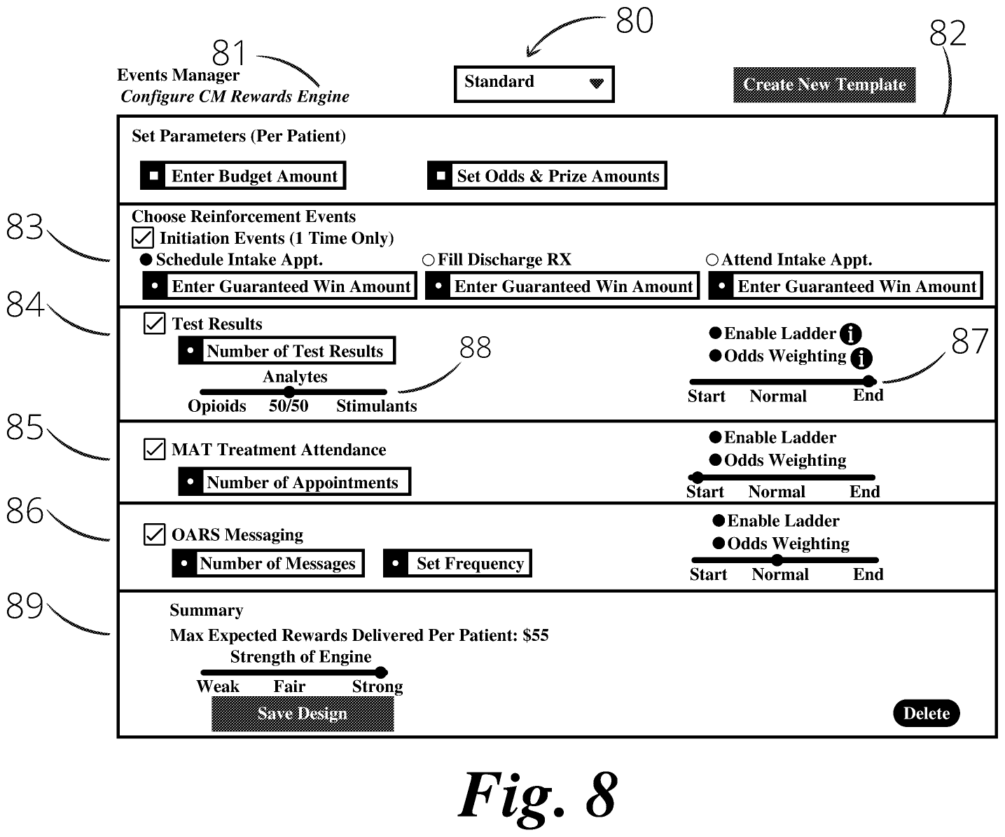

FIG. 8 provides a perspective view of an embodiment of a user interface 80 for configuring a contingency management rewards engine 81. The contingency management engine 81 enables the design and deployment of a contingency management program around a number of variables. In this embodiment, some of the variables to be configured in the contingency management engine 81 are the general parameters 82 for the contingency management program, including, but not limited to, a budget, odds, and prize amounts per patient. The engine 81 is also configurable to set a number of different targeted behavioral events for reinforcement. In the embodiment shown in FIG. 8, these events include initiation events 83, such as scheduling intake appointments, filling discharge prescriptions, and attending intake appointments. In this embodiment, the initiation events 83 can be set as guaranteed wins, and, if the events 83 are set as guaranteed wins, the engine 81 is operable to deliver a guaranteed reward amount upon the occurrence of any one of the events 83. The purpose of setting certain events, particularly, in this embodiment, initiation events 83, as guaranteed wins is because such events may occur a very limited number of times and it is important to positively reinforce such behaviors when the opportunity presents itself. In other words, the less an event occurs, the more important it may be to guarantee positive reinforcement of said event.

Still referring to the embodiment in FIG. 8, other events that may be targeted for reinforcement by the engine 81 include test result events 84, appointment attendance events 85, and messaging events 86. In this embodiment, for each targetable event other than the initiation events 83, the engine 81 offers a rewards and chances ladder 87 that may be configured to increase, balance, or weigh the chances for or amount of rewards during a period of the contingency management program. For example, in the embodiment shown in FIG. 8, the ladder 87 for test result events 84 is configured to increase the chances for or amount of rewards for analyte negative test results towards the end of the contingency management program. Similarly, the ladder 87 for appointment attendance events 85 is configured to increase the chances for or amount of rewards for patients attending appointments in the beginning of the program. Conversely, the ladder 87 for messaging events 86 is configured to balance the chances for or amount of rewards for patients responding or communicating with administrative or other users throughout the period of the program.

Also shown in the FIG. 8 embodiment is a rewards and chances ladder 88 that may be configured to increase, balance, or weigh the chances for or amount of rewards depending on whether the analyte negative test results are opioid negative test results or stimulant negative test results. For example, in this embodiment, the ladder 88 is configured to balance the chances for and amount of rewards for analyte negative test results, regardless of if the analytes are opioids or stimulants. However, in other embodiments, the ladder 88 may be configured to weigh opioid negative test results more heavily than general stimulant negative test results to the extent that only opioid negative test results may increase any given patient's chances for or amount of rewards.

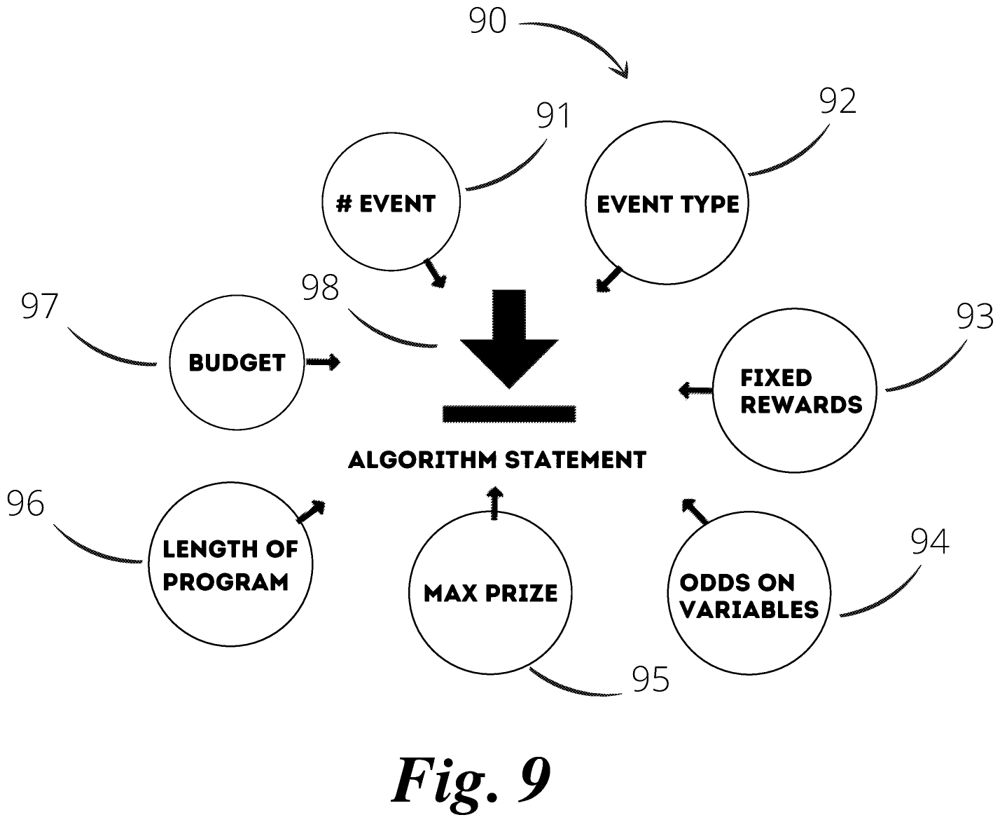

Referring now to both FIGS. 8 and 9, the contingency management system 1 is operable to display a strength indicator 89 for the configured contingency management engine 81. The strength indicator 89 indicates or reveals the strength or effectiveness of the contingency management program to be operated by the engine 81. The system 1 utilizes an algorithm 90 to determine the effectiveness of the program design based on numerous variables that may or may not be independently configurable via the engine 81. For example, in the embodiment shown in FIG. 9, the algorithm 90 determines the efficacy of the program using the following variables: (1) the number of events 91; (2) the type of events 92; (3) whether or not the rewards for positive behavioral events are fixed 93; (4) odds on variables 94, such as odds weighting between the beginning and end of the program; (5) whether or not program games have a maximum prize amount 95; (6) the length of the program 96; and (7) the total budget for the program 97. In this embodiment, after weighing each of the aforementioned variables, the system 1 is then operable to generate an algorithm statement 98, which is displayed as a strength indicator 89 on the user interface 80 for configuring the engine 81.

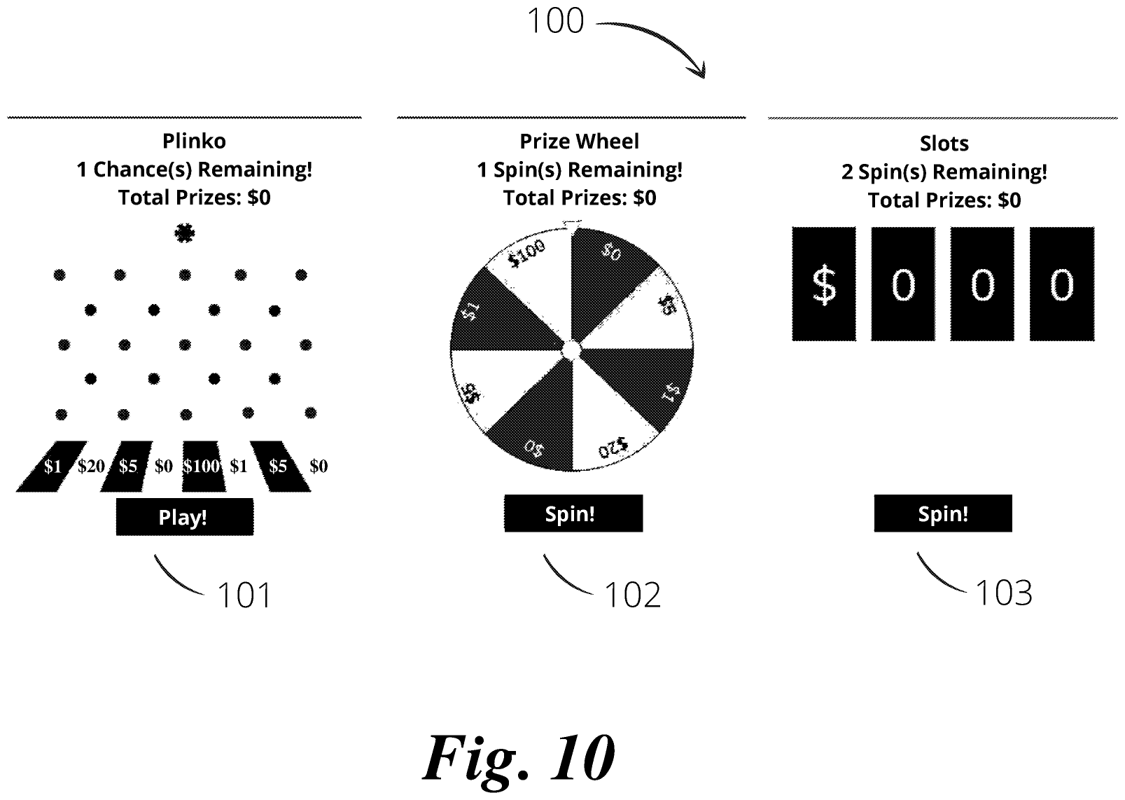

FIG. 10 provides a perspective view of embodiments of user interfaces 100 for games 101, 102, and 103. In these embodiments, the games 101, 102, and 103 are playable on patients' mobile user devices 6. These embodiments illustrate how the contingency management system 1 incorporates gamification to improve the outcome of targeted events by awarding participants or patients the opportunity to earn prizes. For example, in these embodiments, when the system 1 generates rewards chances upon the occurrence of a positive behavioral event, the games 101, 102, or 103, are operable to award prizes based only on the rewards chances generated. When patients are given the opportunity to play games 101, 102, or 103 in order to potentially win prizes as a result of their positive behavior, this may affect the reinforcement of said behavior.

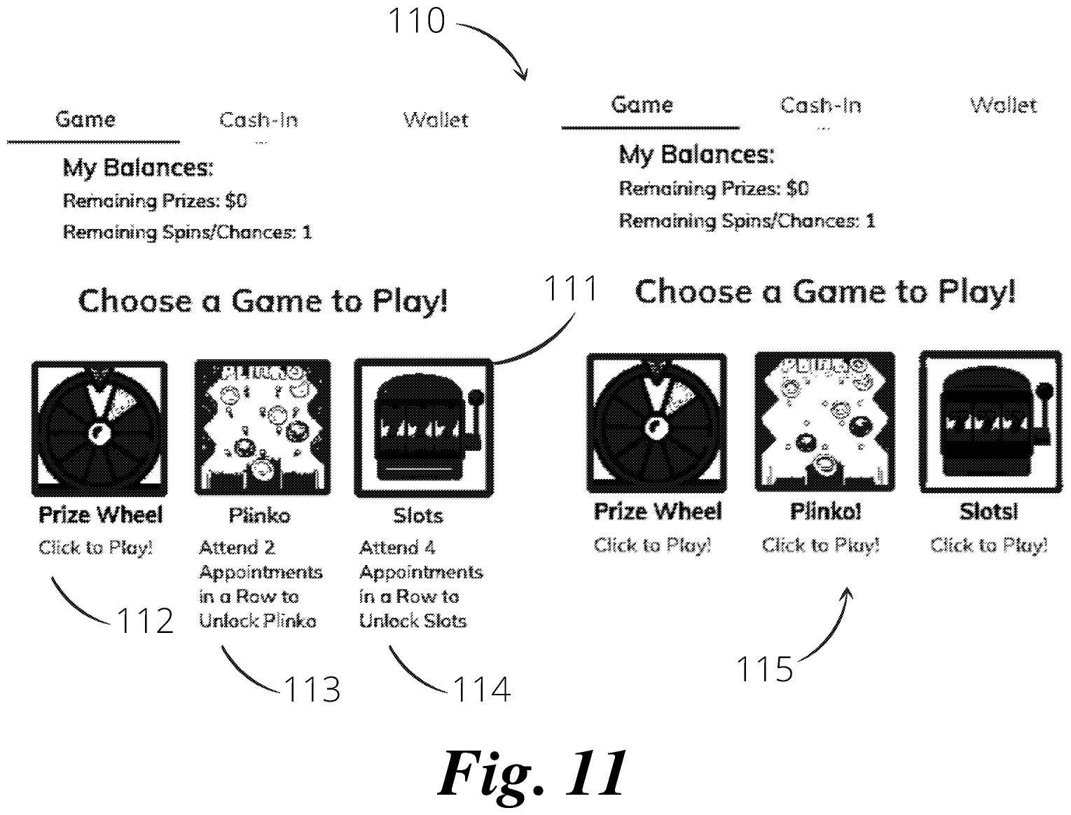

Similarly, FIG. 11 provides a perspective view of embodiments of user interfaces 110 for tiered gaming, which is another example of the incorporation of gamification to improve behavioral outcomes for participants or patients. For example, the first user interface 111 informs patients, who already have the first game 112 unlocked, of the consistent behaviors necessary to unlock the second game 113 and third game 114. In this embodiment, patients' consistent attendance of appointments will unlock the second game 113 and the third game 114, as shown on the second user interface 115. When patients are merely given the opportunity to unlock different games in a tiered gaming system as a result of their positive behavior, this may also affect the reinforcement of said behavior.

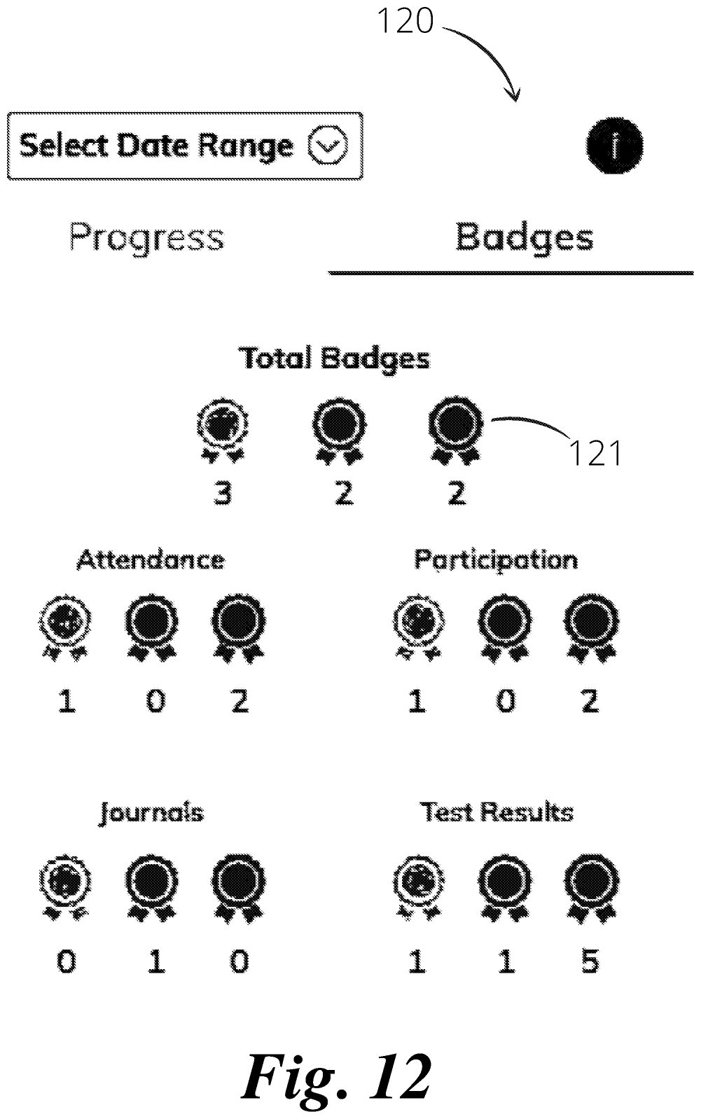

FIG. 12 provides a perspective view of an embodiment of a user interface 120 displaying badge rewards 121, which is another example of how positive behaviors can be reinforced without the use of cash rewards, such as electronic gift cards 32, similar to the example of tiered gaming in FIG. 11. In this embodiment, badge rewards 121 are used instead of cash rewards toward the end of the contingency management program period. However, in other embodiments, badge rewards 121 may be used to supplement cash rewards to increase the length and effectiveness of the program.

While several variations of the present disclosure have been illustrated by way of example in preferred or particular embodiments, it is apparent that further embodiments could be developed within the spirit and scope of the present disclosure, or the inventive concept thereof. It is also to be expressly understood that elements described in one embodiment may be incorporated with any other embodiment in combination with any other elements disclosed herein in the various embodiments. It is also to be expressly understood that any modifications and adaptations to the present disclosure are within the spirit and scope of the present disclosure, and are inclusive, but not limited to the following appended claims as set forth.

What is claimed is:

1. A contingency management system comprising:
a first server;
a contingency management engine, wherein the contingency management engine is stored in at least one of a computer memory, the first server, or a database; and
the computer memory, wherein the computer memory is configured to store program instructions executable by at least one processor, wherein the contingency management engine is operable to conduct the steps of:
sending an event information to a first user device; and
sending a chance for rewards to a second user device.

2. The contingency management system of claim 1 further comprising a second server, wherein the contingency management engine is further operable to conduct the step of sending a validation for the event information from the second server to the first server.

3. The contingency management system of claim 1, wherein the first user device comprises an administrative user device.

4. The contingency management system of claim 3, wherein the administrative user device is operable to display an administrative dashboard on a user interface.

5. The contingency management system of claim 3, wherein the administrative user device is operable to display a user interface for configuring the contingency management engine.

6. The contingency management system of claim 5, wherein the user interface is operable to allow the contingency management engine to be configured to target an event for reinforcement.

7. The contingency management system of claim 5, wherein the user interface comprises a ladder, the user interface being operable to configure the ladder to perform at least one of the following steps: increasing the chance for rewards during a period, decreasing the chance for rewards during the period, or balancing the chance for rewards during the period.

8. The contingency management system of claim 5, wherein the user interface comprises a ladder, the user interface being operable to configure the ladder to perform at least one of the following steps: increasing the chance for rewards based on the event information, decreasing the chance for rewards based on the event information, or balance the change for rewards based on the event information.

9. The contingency management system of claim 1, wherein the second user device comprises a mobile user device.

10. The contingency management system of claim 1, wherein the contingency management engine is further operable to conduct the step of sending a message from the first user device to the first server.

11. The contingency management system of claim 1, wherein the contingency management engine is further operable to conduct the step of sending a message from the first server to the second user device.

12. The contingency management system of claim 1, wherein the contingency management engine is further operable to conduct the step of sending the event information from the first user device to the first server.

13. The contingency management system of claim 12, wherein the event information comprises an appointment information.

14. The contingency management system of claim 13, wherein the contingency management engine is further operable to conduct the step of validating the appointment information by using a geographic information system.

15. The contingency management system of claim 1, wherein the second user device is operable to display a contingency management application on a user interface.

16. The contingency management system of claim 15, wherein the contingency management application is operable to display a game of chance on the user interface.

17. The contingency management system of claim 16, wherein the contingency management engine is further operable to conduct the step of sending a reward to the contingency management application of the second user device upon completion of the game of chance.

18. The contingency management system of claim 17, wherein the contingency management application is operable to conduct the step of redeeming the reward for a prize.

19. The contingency management system of claim 18, wherein the prize is at least one of an electronic gift card or a badge reward.

20. The contingency management system of claim 1, wherein the contingency management engine is further operable to conduct the step of sending an opportunity to play a game to the second user device.
